(19) United States
(12) Patent Application Publication
(10) Pub. No.: US 2006/0105353 A1
(43) Pub. Date: May 18, 2006

(54) Non-invasive prenatal fetal cell diagnostic method

(76) Inventors: Syed M. Jalal, Rochester, MN (US); Harinder Chhabra, Rochester, MN (US)

Correspondence Address: MUETING, RAASCH & GEBHARDT, P.A., P.O. BOX 581415, MINNEAPOLIS, MN, 55458, US

(21) Appl. No.: 10/989,529

(22) Filed: Nov. 16, 2004

(51) Int. Cl. C12Q 1/68; G01N 33/567

(52) U.S. Cl. 435/6; 435/7.21

(57) ABSTRACT

The present invention provides methods for prenatal diagnosis. The method includes distinguishing fetal cells from maternal cells. The fetal cells may be analyzed to measure, for instance, the presence or absence of genetic markers.

### BACKGROUND

Approximately 8 in 1,000 live-born human infants have a major chromosomal abnormality, of which 81 to 95% involve aneuploidy of chromosomes 13, 81, 21, X or Y. These chromosomal abnormalities result in a variety of clinical problems, such as Down syndrome, Turner syndrome, and Klinefelter syndrome. When all sources of genetic problems are considered, there are over 4,000 inheritable genetic diseases, including, for example, Tay-Sachs Disease, Huntington Diseases, Cystic fibrosis, various forms of cancer, Sickle Cell Anemia, Phenylketonuria, and Congenital hypothyroidism. Given the progress in identifying the genetic markers for various genetic diseases, and the increasing use of prenatal diagnosis, there is an ever increasing need for a reliable and non-invasive method of ascertaining the genetic health of a fetus.

The current diagnostic test for chromosomal abnormalities is cytogenetic analysis. This is an accurate and well-established test, but requires either amniocentesis or chorionic villus sampling (CVS) to obtain fetal tissue, both of which are invasive procedures. These procedures suffer from the disadvantages of delay in diagnosis and risk to both the fetus and mother. Amniocentesis is generally done at 15 weeks of gestation, while CVS is done at 9-12 weeks gestation. Early diagnosis is advantageous because of reduced emotional stress for the parents, and the medical advantages associated with early termination, should that be the parents' choice, or providing greater time to prepare for the hardship of raising a child with a genetic disorder. The risk of fetal loss associated with amniocentesis is about 0.5% when done at 16 weeks; however, the level of risk increases for earlier amniocentesis or CVS.

In order to minimize medical costs, screening tests have been in use to identify those pregnant females at risk for fetal chromosome abnormalities. Those at risk then are typically advised to have amniocentesis or CVS-based chromosome analysis. Fetal alpha-feto-protein (AFP) is known to circulate in maternal blood serum of pregnant females. The amount of AFP and other serum markers are used to identify females at risk for trisomy 18 (Edwards Syndrome), trisomy 21 (Down Syndrome) or tetraploidy. However, these screening tests have a projected detection rate of 60%-80% with an average of about 70%, and they are limited to just the chromosome anomalies mentioned above. A more reliable test for fetal numeric anomalies of chromosomes 13, 18, 21, X and Y (also tetraploidy) based on a non-invasive procedure would be a substantial improvement.

Given the problems associated with current invasive prenatal genetic diagnosis techniques and screening methods, a great deal of effort has been invested in researching noninvasive prenatal diagnostic methods utilizing fetal cells and/or nucleic acids from maternal blood samples. It has been discovered that during pregnancy, a variety of cell types of fetal origin cross the placenta and circulate within maternal peripheral blood. The presence of fetal trophoblastic cells circulating in maternal blood has been known since 1959, and the presence of circulating fetal lymphocytes during pregnancy has been known since 1969. It is now understood that there are three principle types of circulating fetal cells: lymphocytes, trophoblasts, and nucleated fetal erythrocytes, and that these cell types are found in small numbers within maternal blood at various times during pregnancy. See for example Holzgreve, et al., “Fetal Cells in Maternal Circulation,” J. Reprod. Med. 37: 410-418 (1992). These circulating fetal cells are a potential source of information on the gender and genetic health of the developing fetus. Unfortunately, the feasibility of using the fetal cells present in maternal circulation for diagnostic purposes is greatly hindered by the relatively scarcity of such cells. Using the Y chromosome as a marker, Krabchi et al. determined that an average of only 2 to 6 fetal cells were present per milliliter of maternal blood. Krabchi et al., Clin. Genet. 60, 145-150 (2001).

Various researchers have investigated the presence and/or use of circulating fetal cells. Reading et al. evaluated a three-stage procedure to use nucleated erythrocytes to determine the number of cells that are fetal rather than maternal in origin. Reading et al., Molecular Human Reproduction v. 1, Human Reproduction v. 10, p. 2510 (1995). Bianchi developed a method for enriching fetal granulocytes and evaluating them by in situ hybridization. Bianchi, U.S. Pat. No. 5,714,325. Fisk et al. purified fetal cells lacking the CD45 marker based on immunophenotyping and the selective adherence of the fetal cells to plastic, followed by expansion of fetal cells in tissue culture. Fisk et al., WO 01/9851 A1. Burchell et al. utilized a method of identifying and isolating embryonic or fetal red blood cells from a sample by binding to one or more adult liver components that are absent from maternal cells. Burchell et al., U.S. Pat. No. 6,331,395. Smith described a method of distinguishing fetal from maternal cells by using a probe complementary to HLA-G mRNA which only hybridized with fetal cells. Smith, U.S. Pat. No. 5,750,339. Thomas provides a method of isolating fetal cells from maternal blood using layered immunosorption to specifically bind erythroid cell precursors. The isolated fetal cells are then permeabilized by detergent and analyzed by fluorescence in situ hybridization. Thomas, U.S. Patent Application No. 2003/0232377 A1.

While there has clearly been extensive research in the field, the development of a non-invasive procedure to diagnose chromosome abnormalities of a fetus from the cells or DNA circulating in maternal blood has been very nearly abandoned due to the technical difficulties involved in distinguishing maternal from fetal cells and the low number of analyzable fetal cells present in maternal blood (Jackson, “Fetal cells and DNA in maternal blood,” Prenatal Diagnosis, 23: 837-846 (2003)). Jackson's review concludes that “although basic work has demonstrated the biologic availability of both fetal cells and their free DNA representatives in the maternal circulation at gestational ages relevant to prenatal diagnosis, much work remains to develop practical technology for their consistent recovery and assay.” In particular, FISH analysis has been hampered by difficulties in cell finding and cell image assessment.

### SUMMARY OF THE INVENTION

The present invention provides a solution to the problems identified above by providing a powerful and reliable method for identifying and characterizing fetal cells in order to conduct a prenatal diagnosis of a fetus. The method can be carried out non-invasively, and can evaluate fetal cells for a much greater variety of types of genetic information than current screening tests.

The present invention provides a method including providing a composition including fetal cells, enriching the composition, and providing an antibody including a first label to the fetal cells under conditions wherein the antibody specifically binds to a fetal cell. The fetal cells may be fetal nucleated red blood cells, and the composition of fetal cells may further include maternal blood cells. The enrichment of fetal cells may be by magnetic cell separation, and may include depletion of cells having CD45 antigen and selecting for cells having CD71 antigen. The method further includes post-fixing the antibody to the fetal cell. The post-fixing may include an aldehyde-based cross-linking fixative such as, for instance, formaldehyde, paraformaldehyde, or paraformaldehyde-picric acid.

The method also includes analyzing the fetal cell. In one aspect, the method includes contacting the fetal cell with a polynucleotide probe including a label under conditions wherein the probe hybridizes to a target polynucleotide sequence in the fetal cell, and detecting the second label in the fetal cell. In another aspect, a target polynucleotide present in the fetal cell is amplified, and the amplified target polynucleotide is characterized. Optionally, the fetal cell is separated from other cells before the amplification.

### BRIEF DESCRIPTION OF THE FIGURES

The file of this patent contains at least one photograph executed in color. Copies of this patent with color photographs will be provided by the Office upon request and payment of the necessary fee.

FIG. 1 shows fNRBCs and other cells after labeling with anti-HbF antibody with AMCA label. FNRBCs are indicated with red lettering and arrows, while WBC are indicated with yellow lettering and arrows. AMCA, aminomethylcoumarin; fNRBCs, fetal nucleated red blood cells; HbF, fetal hemoglobin; and WBC, white blood cells.

FIGS. 2A and 2B show male fNRBCs labeled with anti-HbF antibody with AMCA label. FNRBCs are indicated with red lettering and arrows, while WBC are indicated with yellow lettering and arrows. FIG. 2A shows cells that have been hybridized with fluorescent polynucleotide probes to chromosome X (green), chromosome Y (orange) and chromosome 18 (aqua). FIG. 2B shows cells that have been hybridized with fluorescent polynucleotide probes to chromosome 13 (green) and chromosome 21 (orange).

FIG. 3 shows normal male fNRBCs labeled with anti-HbF antibody with AMCA label and subjected to FISH, as in FIG. 2B, but at greater magnification.

FIGS. 4A and 4B show normal female fNRBCs labeled with anti-HbF antibody with AMCA label. FNRBCs are indicated with red lettering and arrows. FIG. 4A shows cells that have been hybridized with fluorescent polynucleotide probes to chromosome X (green) and chromosome 18 (aqua). FIG. 4B shows cells that have been hybridized with fluorescent polynucleotide probes to chromosome 13 (green) and chromosome 21 (orange).

FIGS. 5A and 5B show fNRBCs of a suspected Turner syndrome fetus labeled with anti-HbF antibody with AMCA label. FNRBCs are indicated with red lettering and arrows. FIG. 5A shows cells that have been hybridized with fluorescent polynucleotide probes to chromosome X (green), chromosome Y (orange) and chromosome 18 (aqua). FIG. 5B shows cells that have been hybridized with fluorescent polynucleotide probes to chromosome 13 (green) and chromosome 21 (orange).

FIGS. 6A and 6B show fNRBCs of a suspected Down syndrome fetus labeled with anti-HbF antibody with AMCA label. FNRBCs are indicated with red lettering and arrows. FIG. 6A shows cells that have been hybridized with fluorescent polynucleotide probes to chromosome X (green), chromosome Y (orange) and chromosome 18 (aqua). FIG. 6B shows cells that have been hybridized with fluorescent polynucleotide probes to chromosome 13 (green) and chromosome 21 (orange).

### DETAILED DESCRIPTION OF PREFERRED ASPECTS OF THE INVENTION

The invention relates to a method of prenatal diagnosis using fetal cells. “Prenatal diagnosis,” as used herein, refers to determining the presence of a disease or condition in a fetus prior to the time when that fetus is born. The method of the invention identifies fetal cells of interest by distinguishing them from maternal cells present in the sample, allowing the identified fetal cells to be analyzed using various techniques. For instance, the method provides an approach to detect fetal conditions or disorders by analysis of fetal polynucleotides. For example, the invention provides a means for determining the sex of the fetus.

As used herein, the term “polynucleotide” refers to a polymeric form of nucleotides of any length, either ribonucleotides or deoxynucleotides, and includes both double- and single-stranded DNA and RNA. A polynucleotide can be present in a cell identified using the methods described herein, obtained directly from a natural source, or can be prepared with the aid of recombinant, enzymatic, or chemical techniques. A polynucleotide can be linear or circular in topology. As used herein, a “target polynucleotide” is a polynucleotide present in a fetal cell. In addition, unless otherwise specified, “a,” “an,” “the,” and “at least one” are used interchangeably throughout this application and mean one or more than one.

In order to conduct the prenatal diagnosis, a composition including fetal cells may be provided. The composition including fetal cells will typically be a blood sample obtained from a pregnant female. The pregnant female may be any mammal and in particular a mammal of commercial or agricultural importance, or a domesticated mammal. For example, the pregnant female may be a horse, cow, sheep, pig, goat, dog, or cat. In a preferred aspect of the invention, the pregnant female is human.

An advantage of some aspects the present invention is the ability to non-invasively evaluate a composition including fetal cells at an early stage of gestation. “Non-invasive,” as used herein, refers to the use of techniques that do not physically invade the space occupied by the fetus or related tissue such as the placenta. As noted above, the composition including fetal cells will typically be a blood sample. It is preferred that the blood sample be obtained from the pregnant female at an early stage of pregnancy. While the gestation period varies from species to species, it is particularly preferred that the sample be obtained in the first trimester of the pregnancy. There are a number of reasons for obtaining the blood sample containing fetal cells at an early stage during the pregnancy. One reason is that the percentage of fetal cells relative to maternal cells may be higher during the first trimester than later times during the pregnancy. Lim et al., Prenatal Diagnosis, 21, 14 (2001). Termination of pregnancy is also technically easier earlier in gestation with less physical and psychological side effects.

The composition including fetal cells is typically a blood sample obtained from a pregnant female. Preferably, the blood sample is a peripheral blood sample as these are easiest to obtain. However, the composition including fetal cells may be any sample, particularly a fluid, containing fetal cells. For example, the composition including fetal cells may be amniotic fluid, urine, lymphatic fluid, or mucous from the maternal cervix or vagina. The composition including fetal cells may also be a fetal blood sample. The sample is obtained from the pregnant woman using routine procedures available in the art, including, for example, standard venipuncture.

Various types of fetal cells are available in maternal blood circulation for analysis. In particular, lymphocytes, trophoblasts, and fetal nucleated red blood cells are useful for prenatal diagnosis. These three types of fetal cells have different advantages as a result of their inherent properties. For example, fetal lymphocytes can be cultivated with relative ease, and have a large number of available immunological reagents targeted to their cell surface molecules, which have been well characterized. However, fetal lymphocytes have a long half life. Fetal lymphocytes may persist in the circulation of the mother for several years, and may thus introduce an element of uncertainty into analysis during subsequent pregnancies. Fetal trophoblasts form the wall of the blastocyst early in development and aid implantation of the embryo in the uterine wall, and are thus useful candidates for prenatal diagnosis. However, they have a tendency to form multinucleated syncytial cells. These types of cells are not well suited to analysis by, for instance, fluorescent in situ hybridization (FISH) as the many nuclei can complicate analysis of chromosome labeling. On the other hand, fetal trophoblasts may be particularly well suited for DNA amplification due to the abundance of genetic material they provide.

Preferred fetal cells for use in the invention are fetal nucleated red blood cells (fNRBCs). Nucleated red blood cells are extremely rare in adult peripheral blood, making it potentially easier to distinguish fNRBC from the non-nucleated cells. The nuclei of these cells also provide a source of nucleotide sequences that can be used for analysis by various methods. Unlike lymphocytes, fNRBCs do not persist long at all in the blood after pregnancy, preventing the problem of interference with later analysis, and decline in number significantly after week 20 of pregnancy in humans. FNRBCs are present at optimal levels between week 13 and 17 of pregnancy in humans. Finally, the cell surface markers of fNRBCs have been well characterized. Their immunophenotype is similar to stromal cells isolated from first trimester liver cells, and they are readily bound by antibodies to the gamma chain of fetal hemoglobin (HbF) and the transferring receptor (CD71).

As fetal cells are present in relatively low quantities in maternal blood, the composition including fetal cells is preferably enriched. “Enrichment” is defined herein as increasing the relative concentration of fetal cells to maternal cells in a sample, preferably while maintaining a high yield of fetal cells. Preferably, cells are enriched by removal of 70%, 80%, 90%, or 99%, preferably, 99% of non-fetal cells. A variety of methods exist for fetal cell enrichment. For example, fetal cells may be enriched from maternal fluid samples by density gradient centrifugation, fluorescence-activated cell sorting (FACS), magnetic activated cell sorting, antibody-conjugated columns, and charge flow separation. Preferably, fetal cells are enriched through the use of magnetic activated cell sorting.

Various media suitable for use in cell separation are known and can be used in the present invention. For instance, density gradient centrifugation can be conducted using media designed for the separation of blood cells, such as the medium available under the trade designation FICOLL-PAQUE (Amersham Biosciences, Piscataway, N.J.). Buffers that could result in lysis of fetal blood cells other than fNRBC, for instance, ammonium/chloride/potassium lysing buffer, are typically not used.

Magnetic activated cell sorting is a separation technique using magnetic labeling to separate target cells from a sample. Antibody or another specific binding compound bearing a magnetic label is bound to a target cell, thereby allowing the cell to be trapped as it passes through a magnetic field. Other specific binding compounds such as lectins (including wheat germ agglutinin and soy bean agglutinin), growth factors, and cytokines may be used in place of antibodies. In direct labeling, the antibody that binds to the target cell is coupled directly to a magnetic particle. For indirect labeling, a second compound, for instance, an antibody, can be used. The second antibody is coupled to magnetic particle while the antibody that binds to the target cell is not. The second antibody is specific for the first antibody, thereby providing the magnetic label indirectly by binding to the first antibody that is bound to the target cell. In addition to the direct vs. indirect distinction, there are two basic types of enrichment; positive selection and depletion. In positive selection, magnetically labeled antibody is bound to the target cells, and then these cells are trapped in the magnetic field. Positive selection can be used for purifying rare cells and can be done relatively rapidly. Depletion, on the other hand, takes the opposite approach, and uses magnetically labeled antibody that is specific for a cell that is to be removed. As the sample is run and exposed to a magnetic field undesired cells are trapped in the field. A depletion strategy is preferred if no specific antibody is available for target cells or antibody binding to target cells is undesirable, and is a useful precursor to positive selection. Any combination of positive selection and depletion and direct or indirect labeling may be used to purify desired fetal cells for the method of the present invention.

When the present invention includes enrichment by positive selection, depletion, or the combination thereof, antigens allowing the separate targeting of desired cells and undesired cells are typically used. For the present invention, antigens may be chosen that can be used to distinguish fetal cells from maternal cells. Examples of such antigens include, for instance, CD45 (typically present on most leukocytes); CD3, CD4, CD5, and CD8 (typically present on T cells); CD12, CD19, and CD20 (typically present on B cells); CD14 (typically present on monocytes); CD16 and CD56 (typically present on natural killer cells); and CD41 (typically present on platelets). Compounds, for instance antibodies, that specifically bind to an antigen can be used. Such antibodies are commercially available, many in a form already conjugated to various types of particles. Caltag (Burlingame, Calif.) and Pharmingen (SanDiego, Calif.) are suitable suppliers of conjugated antibodies, for example. Other specific binding compounds such as lectins (including wheat germ agglutinin and soy bean agglutinin), growth factors, and cytokines may be used.

Alternatively, or in addition, antigens present on fetal cells but not maternal cells may be used for positive selection. For example, in an aspect of the present invention, antibodies directed to CD71 may be used to positively select for fetal nucleated red blood cells. Other antigens found on fetal cells are the thrombospondin receptor (CD36), CD35, CD44, CD55 and glycophorin A. Characterization of antigens expressed on the surface of fetal nucleated red blood cells is described by Alvarez et al., Clin. Chem., 45, 1614 (1999). Other types of fetal cells will have different antigens that can be utilized for positive selection of these cells.

In a preferred aspect of the present invention, fetal nucleated red blood cells are enriched using magnetic activated cell sorting by depletion using CD45 antibody followed by positive selection for CD71 antigen. A detailed discussion of fNRBC enrichment by magnetic activated cell sorting is provided by Reading et al, Molecular Human Reproduction v. 1, Human Reproduction v. 10, p. 2510 (1995). The time allowed for incubation using this method is preferably not extended, as monocytes may eventually phagocytize the beads and be isolated along with the cells of interest.

Once fetal cells have been enriched, in some aspects of the invention cells are fixed to a surface such as a slide. Should it be desirable to fix the cells to a surface, a fixative may be used. Fixatives are often used in immunohistochemical analysis to avoid cell decomposition and protect the cells against various deleterious effects involved in the immunohistochemical process. Fixatives may be used at two different times during the method of the invention. First, they may be used to fix cells to a surface for analysis. Later, they may be used to conduct a post-fix after fetal cells have been labeled. Post-fixing is described herein. Generally, but not necessarily, different fixative solutions are used for the first fix and the post-fix. See Kiernan, J. A., Histological and Histochemical Methods: Theory and Practice, 3^{rd} Ed.(2001) for general fixative procedures.

A large number of fixatives are available. Fixatives used to facilitate antibody-antigen recognition include formaldehyde, paraformaldehyde, paraformaldehyde-picric acid, glutaraldehyde, mercuric chloride, periodate-lysine-paraformaldehyde, and precipitating fixatives such as ethanol, methanol, and acetone. To determine the appropriate fixative for a particular system, guidelines are available that indicate the suitability of a particular fixative for particular antigens. For example, formaldehyde, paraformaldehyde, and paraformaldehyde-picric acid are typically recommended to use with most proteins, peptides, and enzymes with low molecular weight, whereas glutaraldehyde is recommended for use with small molecules such as amino acids.

In order to distinguish fetal cells from maternal cells, an antibody or other compound that specifically binds to fetal cells but not to maternal cells can be used. As used herein, the phrase “specifically binds” refers to a compound, for instance an antibody, that will, under appropriate conditions, interact with a specific molecule even in the presence of a diversity of potential binding targets. With respect to an antibody, “specifically binds” means the antibody interacts only with the epitope of the antigen that induced the synthesis of the antibody, or interacts with a structurally related epitope. The bound compound may be detected directly or indirectly.

Preferably, an antibody or other specific binding compound includes a label. As used herein, the term “label” refers to a compound that permits the detection of the antibody. Typically, when an antibody includes a label, the label is covalently attached to the antibody. Examples of such compounds include, for instance, fluorescent compounds (e.g., green, yellow, blue, orange, or red fluorescent proteins and non-proteins), aminomethylcoumarin, fluorescein, luciferase, alkaline phosphatase, and chloramphenicol acetyl transferase, and other molecules detectable by their fluorescence or enzymatic activity. Other examples of such compounds include biotin and other compounds that permit the use of a secondary compound that includes a detectable compound. Methods for the covalent attachment of label to an antibody or other specific binding compounds are routine and known to those skilled in the art. Attachment may be conducted by one skilled in the art, or antibodies conjugated to label may be obtained commercially from a suitable company (e.g. Molecular Probes, ALT, Quantum Dot)

“Antibody,” as used herein, includes human, non-human, or chimeric immunoglobulin, or binding fragments thereof, that specifically bind to an antigen. In particular, antibodies may be used to identify fetal cells by binding to target antigens present primarily on fetal cells. Suitable antibodies may be polyclonal, monoclonal, or recombinant, or useful fragments such as Fab. Methods of preparing, manipulating, labeling, and using antibodies are well known in the art. See, e.g., Current Protocols In Molecular Biology, Greene Publishing and Wiley-Interscience, edited by Ausubel et al., including Supplement 46 (April 1999). Many suitable antibodies are available commercially, see, e.g., Cortex Biochem, Inc. (San Leandro Calif.), Becton-Dickinson Immunocytometry Systems (San Jose, Calif.), Pharmingen (San Diego Calif.), Caltag Laboratories, Inc. (Burlingam Calif.), DAKO Corp. (Carpinteria Calif.).

While numerous antibodies and compounds exist that can potentially be used to specifically bind to fetal cells, antibodies are typically chosen that exhibit minimal binding to antigens present on maternal cells so that they do not interfere with interpretation of later analysis by techniques such as FISH or PCR. For example, in one aspect of the invention, a monoclonal antibody including a label may be used to identify the cytoplasm of fNRBCs. Cells may be labeled for immediate analysis, or may be used to provide samples that are mailed or otherwise transported for subsequent analysis. Should samples not be used immediately, the label used may fade with time.

As noted above, those skilled in the art will understand that antibodies are preferably chosen that do not interfere with interpretation of later analysis. For example, when a commercially available mouse monoclonal IgG1 antibody to human fetal hemoglobin (HbF) conjugated to fluorescein isothiocyanate (FITC) (purchased from Caltag Laboratories (Burlington, Calif.)) was used to label fNRBC cytoplasm the cytoplasm was successfully labeled but the green labeled FISH probes for specific chromosomes were obscured by the green of the FITC of the antibodies bound to the cytoplasm.

Antibody binding to cells is carried out using procedures known to those skilled in the art. Typically, if cells are fixed on a slide, the slide is dried and then antibody, diluted in buffer, is added. The slides are then incubated for a specified time, typically at room temperature. After incubation, the slides are washed with buffer to remove unattached antibody. Antibody can also be bound to cells in solution, for example, using techniques such as those used by those skilled in the art for flow cytometry. Typically, the antibody used also includes a label.

Subsequent to labeling fetal cells with antibody, a fixative may be used to carry out a “post-fix”. “Post-fix,” as used herein, refers to the application of a fixative to cells already labeled with, for example, an antibody. To distinguish the fixative used in this step from the fixative that is used to secure cells to a surface, the post-fix fixative is referred to herein as the “cross-linking fixative”. While not intending to be bound by theory, the post-fix appears to function in the method of the present invention by reinforcing the binding of antibody to fetal cells, thereby making it easier to distinguish fetal cells from maternal cells that may also bear antibody bound with lower affinity.

A large number of cross-linking fixatives are available. Cross-linking fixatives include formaldehyde, paraformaldehyde, paraformaldehyde-picric acid, glutaraldehyde, mercuric chloride, and periodate-lysine-paraformaldehyde. Other cross-linking fixatives include 4-azidobenzoic acid (3-sulfo-N-succinimidyl) ester sodium salt (Sulfo-HSAB), 1,4-Bis [3-(2-pyridyldithio) propionamido] butane (DPDPB), Bis [2-(4-azidosalicylamido)ethyl] disulfide (BSOCOES), Bis [2-(4-azidosalicylamido)ethyl] disulfide (BASED), Dimethyl 3,3′-dithiopropionimidate dihydrochloride (DTBP), Ethylene glycol disuccinate di(N-succinimidyl) ester (EGS), and Sebacic acid bis (N-succinimidyl) ester (DSS), each of which is commercially available from, for instance, Sigma-Aldrich (St. Louis, Mo.). Aldehyde-based cross-linking fixatives, such as formaldehyde, paraformaldehyde, paraformaldehyde-picric acid, glutaraldehyde, and periodate-lysine-paraformaldehyde are preferred, with formaldehyde being the most preferred. Typically the cross-linking fixative is used in a 0.1% to 10% solution, by volume. Most preferably, a 1% solution of formaldehyde (aq.) is used for post-fixing antibody to fetal cells. Preferably, a cross-linking fixative is not ethanol or a mixture of ethanol and glacial acetic acid. Cells are post-fixed by incubating them in the post fix solution, after which the post-fix solution is washed away.

Once fetal cells have been distinguished from maternal cells, for instance by antibody labeling, the fetal cells can be characterized in order to identify traits such as gender or chromosomal or genetic abnormalities. Typically, the characterization is based on the genomic DNA present in the fetal cell, and is referred to as genetic characterization. The genomic DNA, or cell genotype, may be characterized through the use of polynucleotide probes or polynucleotide primers that interact with a target polynucleotide present in the fetal cells, as in FISH or PCR. Other suitable means for identifying sequences of interest such as mutations or polymorphisms may be utilized, such as sequencing of the DNA at portions of interest, differential hybridization to wild-type or mutant sequences, denaturing or non-denaturing gel electrophoresis following digestion with appropriate restriction enzymes, conventional restriction fragment length polymorphism assays, or microarray techniques, for example.

Any information that can be provided by analysis of the genetic information of a particular organism may be provided by analysis of fetal DNA. This may be the identification of various normal phenotypes or gender. Chromosomal or genetic abnormalities may also be identified. Chromosomal abnormalities include abnormalities in particular chromosomes or the number of chromosomes, and are predictive of various disorders. For example, trisomy in chromosome 21 is indicative of Down syndrome, an XXY trisomy is indicative of Klinefelter syndrome, a single X chromosome is indicative of Turner syndrome, and trisomy at chromosomes 13 or 18 is indicative of Patau syndrome or Edwards syndrome, respectively. Chromosomal translocations and deletions, and the detection of various mutations (deletions, insertions, transitions, transversions, and other mutations) can be detected by DNA analysis as well.

Genetic disorders detectable at the nucleotide sequence rather than chromosome level can be detected as well. For example, other genetic disorders detectable by DNA analysis include 21-hydroxylase deficiency or holocarboxylase synthetase deficiency, aspartylglucosaminuria, metachromatic leukodystrophy, Wilson's disease, steroid sulfatase deficiency, X-linked adrenoleukodystrophy, phosphorylase kinase deficiency, and type III glycogen storage disease. Essentially any genetic disease where the gene has been cloned and mutations detected can be identified in fetal cells by aspects of the present invention. Polynucleotide probes useful for genetic characterization by identification of a target polynucleotide are commonly available, or can be designed by the skilled artisan using methods routine and known in the art. One skilled in the art will understand that the genetic basis of other disorders are being discovered on an ongoing basis, and at an increasing rate, especially in view of the recent sequencing of the human genome. Accordingly, the present invention is not limited by the types of genetic disorders that have been currently listed or that can currently be identified.

Cell preparations made according to the method of the invention are well suited for evaluation by in situ hybridization (ISH), with FISH analysis being particularly preferred. In situ hybridization (ISH) refers to hybridization of a polynucleotide probe to a target polynucleotide, such as chromosomal DNA or mRNA in a cell. The probe may be RNA, DNA, a peptide nucleic acid, a chimeric nucleic acid such as a DNA-RNA chimera, or the like. For ISH analysis, non-fluorescent labels such as biotin or digoxigenin may be used, and then visualized using reagents such as fluorochrome-conjugated avidin or streptavidin. When FISH analysis is done, a variety of fluorescent labels may be used, such as fluorescein, SPECTRUMORANGE™, SPECTRUMGREEN™, SPECTRUMRED™, and others known in the art. A variety of variants of the FISH technique are well known and may be used in accordance with the invention, such as M-FISH, Poly-FISH, PRINS, and interphase FISH.

In situ hybridization techniques are well known in the art, and is described generally in Choo, ed., In Situ Hybridization Protocols, Methods in Molecular Biology, Vol. 33 (1994). In situ hybridization is typically carried out by prehybridization of the target cells to increase accessibility of the target DNA or RNA, hybridization of one or more polynucleotide probes to the nucleic acid in the target cell, posthybridization washes to remove nucleic acid fragments not bound during hybridization, and finally detection of the hybridized nucleic acid fragments.

The method of detection of probe hybridized to target DNA of fetal cells depends on the specific compounds used as a label. A microscope is generally used to detect a precipitate or dye such as a fluorophore where it is bound within the cell. It is often useful to utilize automated data collection and image analysis. FACS analysis may be used for example if the labeled cells remain in solution. When viewing the hybridized probe or probes, it is preferable that the antibody, for instance, anti-HbF, used to label fetal cells is also visible, as this allows simultaneous characterization of the cell genotype with confirmation that fetal cells are being viewed. For example, an aspect of the invention using antibody to HbF with a blue fluorophore will highlight fetal cell cytoplasm as blue, while fluorescent probes to various chromosome locations will characterize the chromosome of the fetal cells being viewed. As these two different labels are generally viewed simultaneously, it is preferable that they be of different colors or emission spectra to avoid interference with one another.

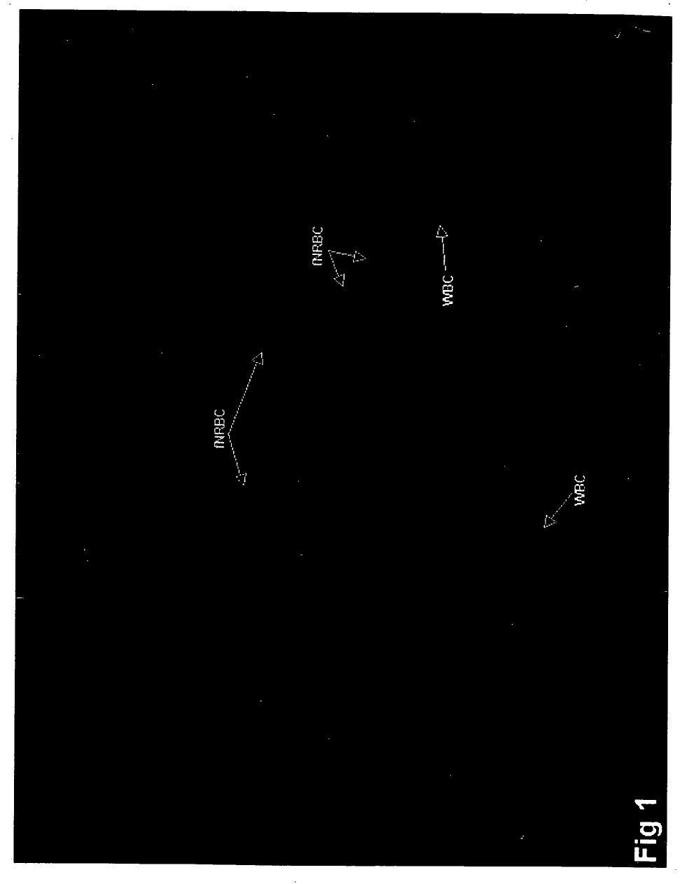

The visualization of the results of one aspect of the method of the present invention are provided in color photographs included with the present application, and identified as FIGS. 1, 2a, 2b, 3, 4a, 4b, 5a, 5b, 6a, and 6b. These photographs illustrate the results obtained when slides bearing cells with fluorescent blue antibody to fetal hemoglobin and fluorescently-labeled hybridization probes were analyzed under an incident-light fluorescent microscope equipped with a 100-watt mercury lamp with a 100× oil immersion objective. The probe set included centromeric-specific α-satellite probes for chromosomes 18 (aqua), X (green), and Y (orange), and locus-specific probes for chromosomes 13 (green) and 21 (orange). The antibody specific to fetal hemogloblin had a blue fluorescent label, illuminating the cytoplasm of fetal cells.

FIG. 1 shows a slide with numerous cells, the majority of which are a pale indigo blue, and generally roughly circular in shape, but lacking a dark center. These are fetal erythrocytes that lack a nucleus and therefore lack the dark center caused by displacement of cytoplasm by the nucleus that is shown in fetal nucleated red blood cells. Also seen in the figure are about a dozen fetal nucleated red blood cells, visible as pale indigo blue circles with dark circular centers. A number of white blood cells (WBC) are also visible. These are irregular shapes and are much darker than either of the other two cell types, and appear to be multinucleated.

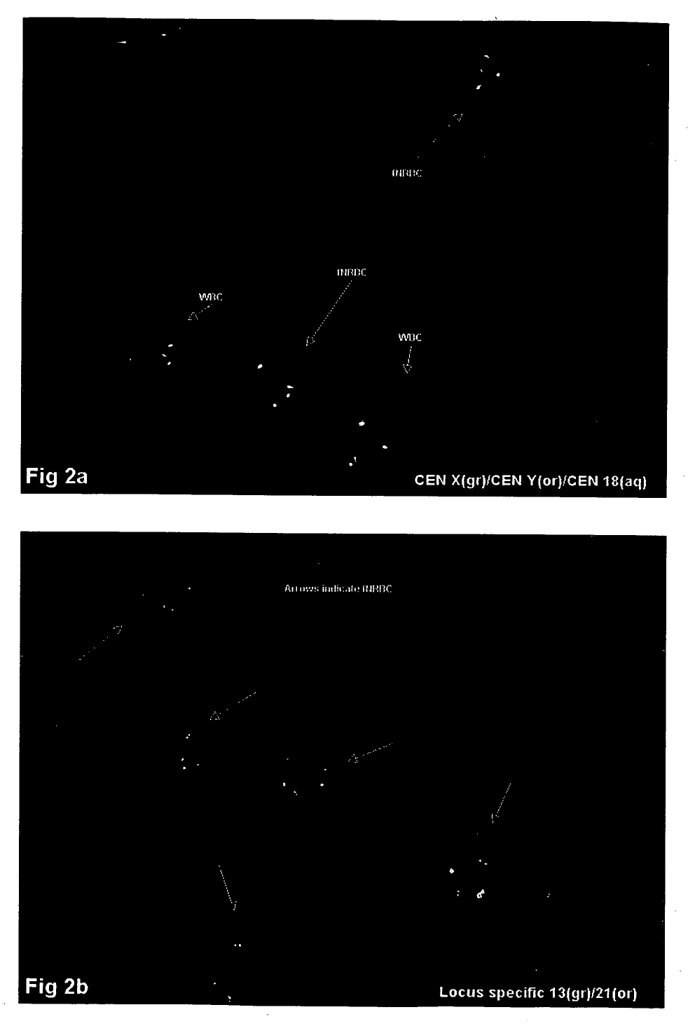

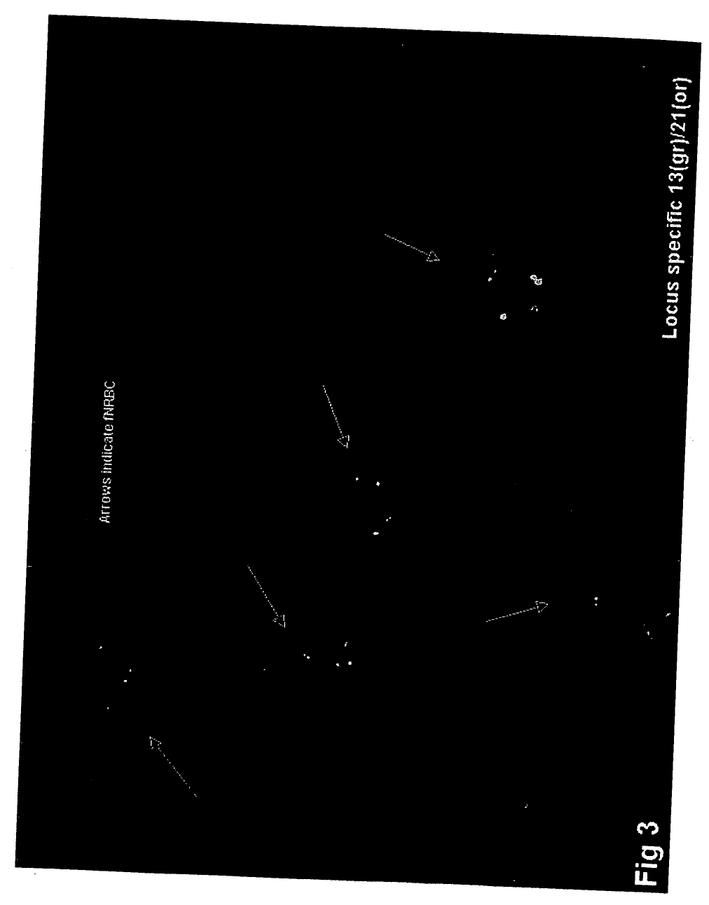

FIG. 2a shows normal male fetal cells with fluorescently labeled cytoplasm amidst several erythrocytes lacking nuclei. The male cells show a single green probe label (X chromosome), a single orange label (Y chromosome), and double aqua labels (chromosome 18). White blood cells are also visible that lack labeled cytoplasm but exhibit the same probe labeling shown for male cells. Labeling of male cells with locus-specific probes is shown in FIG. 2b, which shows pale indigo blue cytoplasm surrounding a dark nucleus with double green probes (chromosome 13) and double orange probes (chromosome 21). FIG. 3 repeats what is shown in FIG. 2b, but at greater magnification.

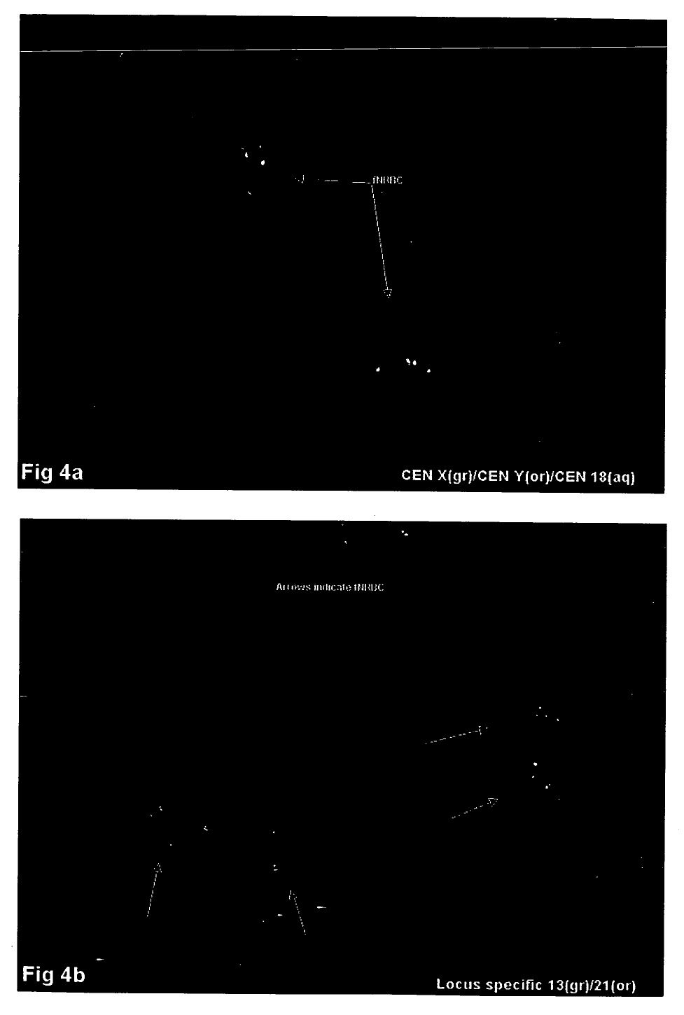

FIG. 4a shows normal female fetal cells with fluorescently labeled cytoplasm, again amidst several erythrocytes that are fluorescently labeled but lack nuclei. The female cells show double green probe labels (X chromosome) and double aqua labels (chromosome 18). Use of locus-specific probes is shown in FIG. 4b, which shows pale indigo blue cytoplasm surrounding a dark nucleus with double green probes (chromosome 13) and double orange probes (chromosome 21).

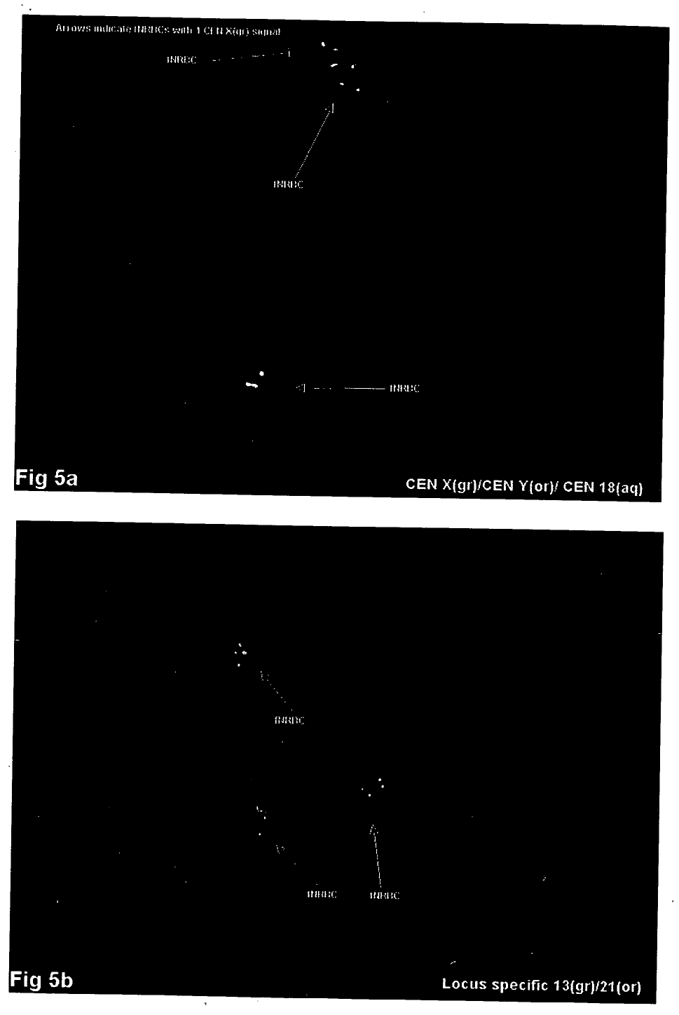

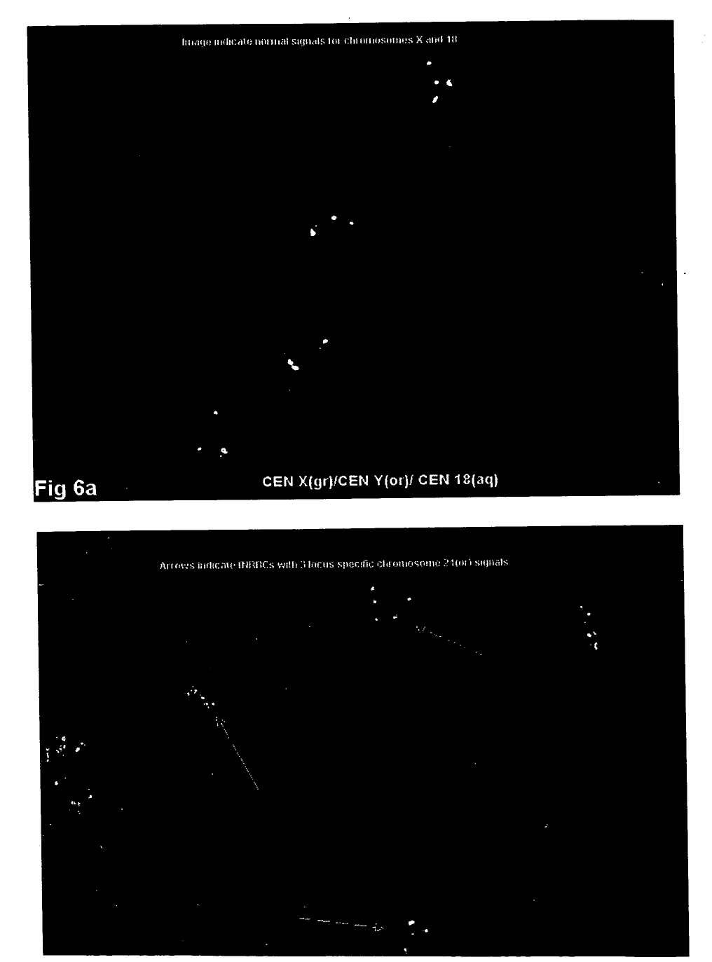

FIG. 5a shows the fetal cells from a suspected Turner Syndrome fetus. These cells showed one green, and two aqua signal from labeled centromeric probes, indicating a single X chromosome (consistent with Turner syndrome), and two chromosome 18s. The results of locus-specific probes are shown in FIG. 5b, which shows two green probes (chromosome 13) and two orange probes (chromosome 21). Fetal cells from a case suspected to have Down Syndrome are shown in FIGS. 6a and 6b. FIG. 6a shows fetal cells with two green centromeric probes (X chromosome) and two aqua centromeric probes (chromosome 18) signals, while FIG. 6b shows three orange signals for chromosome 21 (consistent with Down syndrome) and two green signals for chromosome 13. All of the figures for the Turner Syndrome and Down Syndrome babies also showed pale indigo blue cytoplasm with a dark nucleus for fetal nucleated red blood cells, amidst a number of pale indigo blue cells lacking a nucleus.

As noted earlier, DNA amplification methods such as PCR provide a suitable alternative to the use of polynucleotide probes for genetic characterization of fetal cells distinguished by antibodies or other compounds specific for fetal cells. For PCR analysis, fetal cells are typically bound to antibodies such as anti-HbF in a fluid environment to allow identification and removal of labeled fetal cells by, for instance, micropipette. Removed fetal cells are preferably then diluted in a buffered solution and individual labeled fetal cells can then be used for PCR amplification.

If DNA amplification, for instance PCR, is used to analyze fetal DNA, the isolated fetal cells are typically lysed to release the fetal DNA, and a target polynucleotide can then be amplified using an appropriate number of cycles of denaturation and annealing. Multiplex amplification may also be used if it is desired to amplify more than one fetal gene simultaneously. Upon amplification, the amplification product is a mixture that contains amplified target fetal DNA, as well as other undesired DNA sequences. The amplified target fetal DNA is then separated from the undesired DNA by known techniques such as gel electrophoresis. Optionally, subsequent analysis of the target fetal DNA may be carried out using further known techniques such as digestion with restriction endonuclease, ultraviolet visualization of ethidium bromide stained agarose gels, DNA sequencing, or hybridization with a labeled DNA probe. Primers corresponding to the sequences flanking sequences of interest for various genetic diseases for use in PCR are known and are either available or can be readily synthesized.

The method of the present invention may also be carried out using a kit. In one aspect, useful kits may include, packaged together in a container, one or more of the following reagents: a labelled antibody that specifically binds to fetal cells, a post-fix solution, at least one polynucleotide probe suitable for ISH or DNA amplification; and other reagents useful for the practice of the method described herein. The components are in a suitable packaging material in an amount sufficient for at least one assay. Instructions for use of the packaged components are also typically included.

As used herein, the phrase “packaging material” refers to one or more physical structures used to house the contents of the kit. The packaging material is constructed by well known methods, preferably to provide a sterile, contaminant-free environment. The packaging material has a label which indicates that the components can be used to genetically characterize fetal cells. In addition, the packaging material contains instructions indicating how the materials within the kit are employed to genetically characterize fetal cells. “Instructions for use” typically include a tangible expression describing the reagent concentration or at least one assay method parameter, such as the relative amounts of reagent and sample to be admixed, maintenance time periods for reagent/sample admixtures, temperature, buffer conditions, and the like.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Enrichment of fNRBCs from Umbilical Cord

An enriched fetal cell population is generally prepared before fetal cells may be genetically evaluated. About 30 ml per patient of PEB buffer (Phosphate Ethylene Diamine Tetraacetic Acid Buffer: 250 mg BSA, 100 μl 0.5M EDTA and PBS 500 ml) was prepared. The volume of blood to be processed was then calculated, and umbilical cord blood was diluted in 1 part PBS (phosphate buffered saline) and 1 part blood. FICOLL-PAQUE (5 ml) (Amersham Biosciences, Uppsala, Sweden) was placed in a separate tube. Diluted sample (3-5 ml) was then gently layered on top of the FICOLL. The sample was then spun for 30 minutes at 1500 rpm at room temperature. The top of the fluid was then aspirated, and the mononuclear cell (MNC) layer was carefully removed into a clean tube. Next, 8-10 ml PBS was added and the sample was spun for 10 minutes at 1500 rpm. Pellets were pooled and re-suspended in 200 μl of PEB buffer, providing total counts of no more than 10⁸ cells/ml.

Magnetic activated cell sorting was then used to purify fetal nucleated red blood cells from other cells present in the blood sample. Undesired cells were first removed using CD45 depletion. CD45 microbeads (40 μL) (MACS, Miltenyi Biotech, Auburn, Calif.) were added to the sample, and the sample was then allowed to incubate for 15 minutes at room temperature. Three 15 ml tubes were then labeled for each patient, using the labels 1) Waste, 2) CD 45 positive, and 3) CD 45 negative. During the incubation, an LS column (MACS, Miltenyi Biotech, Auburn, Calif.) was prepared by washing with 3 ml of PEB, which was eluted into the waste tube. The LS column was then placed in the magnet. PEB (3 ml) was then added to each sample, and the samples were spun at 1500 rpm for 5 minutes. The sample was then re-suspended in 1 ml PEB and applied to the LS column. A clean tube labeled ‘CD 45 negative’ was placed under the column to collect the cells lacking CD45 antigen, as CD 45+ cells are retained in the column. After the cell preparation had completely entered the column, 3 ml PEB was added and allowed to drain through. The addition of PEB was repeated 2 times. The tube and the LS column were then removed from the magnet assembly. CD45+ cells can optionally be collected if desired at this point. The ‘CD 45 negative’ tube was then spun at 1200 rpm for 5 minutes, after which the cells were re-suspended in 1 mL PEB. The cells were then counted on a Coulter ACT 10. Cells were again re-suspended to yield total counts of 10⁷ cells/ml. Positive selection for fetal cells was then conducted using a magnetic cell separator and anti-CD71 antibody, in which the sample that previously had been depleted of CD45+ cells was now positively selected for CD71 cells. For the first step, 20 μL of CD71 microbeads (MACS, Miltenyi Biotech, Auburn, Calif.) were added to each 100 μL of sample of CD45− cells and incubated for 15 minutes. An MS column (MACS, Miltenyi Biotech, Auburn, Calif.) was then prepared by washing with 0.5 ml of PEB, after which it was then positioned in the magnetic separator. Next, 3 ml PEB was added to the sample, which was then spun for 5 minutes at 1000 rpm. Two 15 ml tubes were then labeled as 1) CD71 positive and 2) CD71 negative. The suspension was then re-suspended in 0.5 ml PEB, and applied to the MS column. The effluent was collected in the tube labeled ‘CD71 negative’. After the cell suspension had completely entered column, 0.5 ml PEB was added and allowed to drain through. PEB addition was repeated twice. The MS column was then removed from the magnet and placed over the “CD71 positive” labeled tube. PEB (1 ml) was then added and allowed to drip through. A further 1 ml PEB was then added and pushed through with a syringe barrel. The effluent contained the CD45−, CD71+ fraction. The CD45−, CD71+ fraction was then spun down and re-suspended in 0.5 ml buffer. To remove the EDTA, RPMI 1640/10% bovine serum albumin (BSA) (3 ml) was added to the sample, which was then spun down. The suspension was then cytospun onto double Cytospin slides using a Shandon Cytospin 4 (Thermo Shandon Limited, Cheshire, United Kingdom) for 5 min at 1000 rpm, and the slides were fixed in 100% EtOH for 5 minutes.

### Example 2

### Marking fNRBCs with HbF Antibody and FISH Analysis

The next step is to label the cytoplasm of nucleated fetal blood cells with an appropriate monoclonal fluorescent antibody in order to distinguish nucleated fetal blood cells from other cells present in the CD45−, CD71+ fraction. The slides, prepared in Example 1, were then aged for 20 minutes at 90° C. in an oven, and then placed in 10 mM phosphate buffer detergent (PBD) for 10 minutes. One liter of PBD solution is 10 mM phosphate buffered saline (PBS) in 990 ml of purified water plus 10 ml of NP-40 detergent. The slides were then air-dried and flooded with 20 μL anti-HbF antibody (Caltag Laboratories, Burlington, Calif.) with a blue fluorophore, AMCA (prepared by Molecular Probes, Eugene. OR), diluted 1:1 with PBS. The slides were then incubated for 1 hour at room temperature. After incubation, the slides were washed in PBD for 2 minutes. The slides were then post-fixed with 1% formaldehyde solution for 10 minutes. Post-fixing is believed to result in greater adherence of the anti-HbF, resulting in brighter fNRBC that are easier to distinguish from other cells. After post-fixing, the formaldehyde was washed off by placing slides in PBD for 2 minutes. The slides are then dehydrated with 70%, 85% and absolute EtOH, for 2 minutes each, at room temperature. After highlighting of the cytoplasm with anti-HbF antibody, FISH was conducted. Centromeric-specific α-satellite probes for chromosomes 18 (SPECTRUMAQUA™), X (SPECTRUMGREEN™), and Y (SPECTRUMORANGE™) were used (AneuVysion Kit CEP® X/Y/18: Vysis, Inc., premixed in hybridization buffer), as well as locus-specific probes for chromosomes 13 (SPECTRUMGREEN™) and 21 (SPECTRUMORANGE™) (AneuVysion Kit LSI® 13/21: Vysis, Inc., premixed in hybridization buffer). Aliquots (10 μL) from each of the probe mixtures were then denatured in the Hybrite (Vysis, Inc.) in a 75° C. water bath for 10 minutes. The Centromeric-specific α-satellite probes (10 μL) were applied to the hybridization area near the slide label, and 10 mL of the locus-specific probes were placed at the other end of each slide. Each hybridization area was then covered with a coverglass (22 by 22 mm) and sealed with rubber cement. The slides were then placed in a humidified chamber at 37° C. overnight.

When hybridization was complete, the rubber cement and coverslip were removed, and the slides were washed in 0.4% standard saline citrate (SSC) for 2 min at 75° C. The slides were then further washed in 1×PBD for 2 minutes at room temperature (25° C.). The slides were then air-dried, and 10 μL of antifade solution VECTASHIELD (Vector Laboratories Inc. Burlingame, Calif.) was applied, along with a coverslip. The slides were then analyzed under an incident-light fluorescent microscope equipped with a 100-watt mercury lamp with a 100× oil immersion objective to aid in analysis. In order to allow simultaneous viewing of signals, dual-pass SPECTRUMORANGE™/SPECTRUMGREEN™ and triple-pass SPECTRUMORANGE™/SPECTRUMGREEN™/SPECTRUMAQUA™ were used. Alternatively, to view individual fluorescences, single-pass SPECTRUMORANGE™, single-pass SPECTRUMGREEN™ and single-pass SPECTRUMAQUA™ can be used. Filters may be provided by Chromatech (Battlebrook, Vt.) or Vysis (Downers Grove, Ill.), for example. Digitized images were generated using a computer based imaging system, (CytoVysion, Vysis Inc) with appropriate software. For each case, at least 2 pertinent nuclei representing each probe set were digitized and printed.

### Example 3

### Diagnostic Results of FISH Analysis

The same probe set, including centromeric-specific α-satellite probes for chromosomes 18 (aqua), X (green), and Y (orange) and locus-specific probes for chromosomes 13 (green) and 21 (orange) used in Example 2 was used to characterize the chromosomes of fNRBC from fetuses. The fNRBCs have a blue fluorescent label in their cytoplasm, making it is easy to identify the centromere or locus specific labels in males or females, and normals or abnormals. Imaging of the FISH labeled slides for normal male cells in FIGS. 2a, 2b, and 3 revealed fNRBC with blue cytoplasm as having a Y (red), an X (green), and two 18 (aqua), while the probe signals on the nuclei of white blood cells present in the sample lacked the prominent blue fluorescently-labeled cytoplasm. Three types of cells present in the sample were thus clearly distinguished; white blood cells had fluorescent probe but no blue cytoplasm, fetal erythrocytes had blue cytoplasm but no nucleus, while only fetal nucleated red blood cells had a nucleus labeled with fluorescent probes and a blue cytoplasm. This technique clearly and specifically identifies fNRBC since white blood cells or any other nucleated cells that might be present lack the blue monoclonal antibody of that labels red blood cell cytoplasm. This provides a way to distinguish fNRBC and only evaluate fluorescent probe binding to this cell type. Imaging of normal female cells, shown in FIGS. 4a and 4b, revealed nuclei with two X (green) and two 18 (aqua) chromosomes, as well as two 13 (green) and two 21 (orange) chromosomes, the latter two being characterized in a separate analysis. Fetal cells from a suspected Turner Syndrome baby had signals for one X (green), and two 18 (aqua), as well as two 13 (green), and two 21 (orange), the latter two again being characterized in a separate analysis. See FIGS. 5a and 5b. The presence of 45,X (Turner Syndrome) in this case was confirmed by conventional FISH and banded chromosome analysis. Fetal cells from a case suspected to have Down Syndrome had two X (green) and two chromosome 18 signals (aqua), but three chromosome 21 (orange) signals and two for chromosome 13 (green). See FIGS. 6a and 6b. The technique described in Examples 1 and 2 and providing the results of the present example was developed and tested on a series of 24 normal and 2 abnormal samples from cord blood samples of newborns, and only four of the normal samples tested did not provide useful results. The accuracy of identification of numeric anomalies of the fetus by this test is above 95% compared to about 70% for screening tests in current use. Direct magnetic microbead CD45 depletion followed by direct microbead CD71 enrichment produced good results, with total depletion of undesired cells of about 99.7%.

### Example 4

### PCR Amplification of Fetal DNA

The detection of chromosomal abnormalities described above can be adapted for PCR amplification of DNA from individual fNRBC. After an enriched supply of the fetal nucleated red blood cells (fNRBC) is isolated from a maternal blood sample (as described in Example 1), the fetal cells have their cytoplasm stained blue by conjugation with monoclonal antibody specific for fetal hemoglobin (HbF). For PCR analysis, the procedure is modified to stain enriched fNRBC with the monoclonal antibody for HbF in a fluid medium (e.g, welled slides). The blue stained fNRBCs are removed by micro-pipette. After removal of stained fNRBCs, the cell suspension is diluted in 10% fetal bovine serum and one or more individual blue fNRBC are micro-pipetted out for polymerase chain reaction (PCR) amplification. PCR is conducted using the procedure originally developed by Saiki (Saiki, Science, 230, 1350-1354), or variations thereof, in which double stranded DNA is separated and annealed to primers chosen to help reveal fetal chromosomal abnormalities or particular undesirable mutations. Thermal denaturation, primer annealing, and polymerase extension are then repeated to amplify the desired DNA to easily detected quantities. Methods for the amplification of DNA from cells, even single cells, are known. See, for instance, Klein et al. (U.S. Pat. No. 6,673,541).

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

What is claimed is:

1. A method of prenatal diagnosis comprising:
providing a composition comprising fetal cells;
enriching the composition;
providing an antibody comprising a first label to the fetal cells under conditions wherein the antibody specifically binds to a fetal cell;
post-fixing the antibody to the fetal cell;
contacting the fetal cell with a polynucleotide probe comprising a second label under conditions wherein the probe hybridizes to a target polynucleotide sequence in the fetal cell; and
detecting the second label in the fetal cell.

2. The method of claim 1, wherein the fetal cells are fetal nucleated red blood cells.

3. The method of claim 2, wherein the composition further comprises a maternal blood cell.

4. The method of claim 3, wherein the enriching comprises magnetic cell separation.

5. The method of claim 2, wherein the composition of fetal nucleated red blood cells is enriched by depletion of CD45 antigen and positive selection of CD71 antigen.

6. The method of claim 1, wherein the antibody is a monoclonal antibody.

7. The method of claim 6, wherein the first label is a fluorescent label.

8. The method of claim 1, wherein the antibody specifically binds to fetal hemoglobin.

9. The method of claim 1, wherein the second label is a fluorescent label.

10. The method of claim 1, wherein the post-fixing comprises an aldehyde-based cross-linking fixative.

11. The method of claim 10, wherein the aldehyde-based cross-linking fixative is selected from the group consisting of formaldehyde, paraformaldehyde, and paraformaldehyde-picric acid.

12. A method of prenatal diagnosis comprising:
providing a composition comprising fetal cells;
enriching the composition;
providing an antibody comprising a first label to the fetal cells under conditions wherein the antibody specifically binds to a fetal cell;
post-fixing the antibody to the fetal cell;
amplifying a target polynucleotide present in the fetal cell; and
characterizing the amplified target polynucleotide.

13. The method of claim 12, wherein the fetal cells are fetal nucleated red blood cells.

14. The method of claim 13, wherein the composition further comprises a maternal blood cell.

15. The method of claim 14, wherein the enriching comprises magnetic cell separation.

16. The method of claim 15, wherein the first label is a fluorescently label.

17. The method of claim 12, wherein the antibody specifically binds to fetal hemoglobin.

18. The method of claim 12, wherein the post-fixing comprises an aldehyde-based cross-linking fixative.

19. The method of claim 18, wherein the aldehyde-based cross-linking fixative is selected from the group consisting of formaldehyde, paraformaldehyde, and paraformaldehyde-picric acid.

20. A method of prenatal diagnosis comprising:
providing a composition comprising fetal cells;
providing an antibody comprising a first label to the fetal cells under conditions wherein the antibody specifically binds to a fetal cell;
utilizing means for improving adherence of the antibody to the fetal cell;
contacting the fetal cell with a polynucleotide probe comprising a second label under conditions wherein the probe hybridizes to a target polynucleotide in the fetal cell; and
detecting the second label in the fetal cell.

21. The method of claim 20, wherein the fetal cells are fetal nucleated red blood cells.

22. A method of prenatal diagnosis comprising:
providing a composition comprising fetal cells;
enriching the composition;
providing an antibody comprising a first label to the fetal cells under conditions wherein the antibody specifically binds to a fetal cell;
post-fixing the antibody to the fetal cell; and
utilizing means for genetically characterizing the fetal cell.

23. The method of claim 22, wherein the post-fixing comprises an aldehyde-based cross-linking fixative.

24. The method of claim 23, wherein the aldehyde-based cross-linking fixative is selected from the group consisting of formaldehyde, paraformaldehyde, and paraformaldehyde-picric acid.
